(19) United States
(12) Patent Application Publication
(10) Pub. No.: US 2025/0025083 A1
(43) Pub. Date: Jan. 23, 2025

(54) METHODS AND SYSTEMS FOR ELECTRICAL AND/OR OPTICAL SIGNAL BASED STRESS MANAGEMENT

(71) Applicant: Augment Me, Inc., Milpitas, CA (US)

(72) Inventor: Sameer Yami, Milpitas, CA (US)

(21) Appl. No.: 18/770,183

(22) Filed: Jul. 11, 2024

Related U.S. Application Data
(60) Provisional application No. 63/527,089, filed on Jul. 17, 2023.

(51) Int. Cl. A61B 5/16; A61B 5/00; G16H 20/70

(52) U.S. Cl. CPC A61B 5/165; A61B 5/0077; G16H 20/70

(57) ABSTRACT

The present disclosure provides automated methods and systems for implementing a pipeline involving the training and deployment of a predictive model for predicting a stress level of an individual and recommending mental exercises for the individual. An exemplary method includes identifying an individual with an unknown stress level, obtaining one or more electrical or optical signals associated with stress from the individual, predicting a stress level of the individual based on the one or more electrical or optical signals obtained from the individual, wherein the stress level of the individual is predicted by a predictive model, and generating a recommendation for the individual based on the predicted stress level, the recommendation including one or more mental exercises for relieving stress for the individual.

### CROSS-REFERENCE TO RELATED APPLICATIONS

This application claims the benefit of and priority to the U.S. Provisional Patent Application No. 63/527,089, filed on Jul. 17, 2023, and titled “METHOD, SYSTEM AND APPARATUS FOR STRESS MANAGEMENT,” the entire content of which is incorporated herein by reference in its entirety.

### TECHNICAL FIELD

The present disclosure relates generally to the field of artificial intelligence technology, and more specifically to automated methods and systems for electrical and/or optical signal-based stress management.

### BACKGROUND OF THE DISCLOSURE

Stress has become a part of modern life that may have negative impacts on physical and mental health. For example, increasing evidence links stress to cardiovascular disease, anxiety, and depression, which may eventually lead to long-term health problems such as heart disease and sudden, catastrophic outcomes such as accidents, injuries, and even fatalities. Therefore, instantly monitoring mental stress is important to prevent accidents or injuries, especially for certain professionals, such as firefighters and smoker drivers due to the life-threatening nature of their jobs.

Current approaches to monitoring mental stress have been focused on questionnaires and professional consultations, which are expensive, time-consuming, and subjective. Additional approaches includes biomarker-based testing, such as testing based on salivary alpha-amylase and cortisol. Although the biomarker-based approach is more objective than the questionnaires and professional consultations, it has drawbacks, such as the requirement of special instruments and the inability to continuously monitor stress.

Additionally, questionnaires and biomarker-based approaches, while providing certain means for measuring stress levels, do not offer the capacity to provide detailed recommendations to relieve the detected stress. Professional consultations, while providing certain recommendations, still are time-consuming and subjective.

Accordingly. there is a need to develop stress monitoring technology for quick. subjective, and automated detection and monitoring of mental stress and for providing instant, detailed, and intelligent recommendations to relieve the detected stress.

### SUMMARY OF THE DISCLOSURE

The present disclosure addresses the above mentioned problems and other problems in the existing stress management by combining advances in machine learning and automated electrical and/or optical signal detection in wearable technology, to develop an automated stress management platform for instant detection of the stress level of an individual and for providing instant recommendations for alleviating the detected stress based on the detected stress level as well as personal information such as personal preference.

Accordingly, the present disclosure provides an automated method for implementing a pipeline involving the training and deployment of a predictive model for predicting a stress level of an individual. An exemplary method includes identifying an individual with an unknown stress level, obtaining one or more electrical or optical signals associated with stress from the individual, predicting a stress level of the individual based on the one or more electrical or optical signals obtained from the individual, wherein the stress level of the individual is predicted by a predictive model, and generating a recommendation for the individual based on the predicted stress level, the recommendation including one or more mental exercises for relieving stress for the individual.

Embodiments disclosed herein also provide an automated system for implementing a pipeline involving the training and deployment of a predictive model for predicting a stress level of individual. An exemplary system includes a processor, and a memory, coupled to the processor, configured to store executable instructions that, when executed by the processor, cause the processor to perform operations including identifying an individual with an unknown stress level, obtaining one or more electrical or optical signals associated with stress from the individual, predicting a stress level of the individual based on the one or more electrical or optical signals obtained from the individual, wherein the stress level of the individual is predicted by a predictive model, and generating a recommendation for the individual based on the predicted stress level, the recommendation including one or more mental exercises for relieving stress for the individual.

The foregoing is a summary and thus contains, by necessity, simplifications, generalizations and omissions of detail; consequently, the summary is illustrative only and is not limiting in any way. Other aspects, inventive features, and advantages of the systems and/or processes described herein will become apparent in the non-limiting detailed description set forth herein.

### BRIEF DESCRIPTION OF THE DRAWINGS

These and other features, aspects, and advantages of the present disclosure will become better understood with regard to the following description, and accompanying drawings, where:

FIG. 1 shows a schematic stress management system for implementing a stress analysis pipeline, in accordance with an embodiment.

FIG. 2 is an example block diagram that depicts the deployment of a stress level predictive model, in accordance with an embodiment.

FIG. 3 is an example block diagram that depicts a process for training a stress level predictive model, in accordance with an embodiment.

FIG. 4 is an example block diagram that depicts a process for deploying a predictive model to predict the stress level of an individual, in accordance with an embodiment.

FIG. 5 is an example block diagram that depicts a process for deploying a predictive model to evaluate potential mental exercise content, in accordance with an embodiment.

FIG. 6 is an example block diagram that depicts a process for recommending one or more mental exercises for an individual, in accordance with an embodiment.

FIGS. 7A-7D illustrate various outputs generated by the disclosed stress management system, in accordance with an embodiment

FIG. 8 is an example block diagram that depicts a process for creating a proper schedule for an individual, in accordance with an embodiment.

FIG. 9 depicts an example computing device for implementing systems and methods described in reference to FIGS. 1-8.

### DETAILED DESCRIPTION

To make the aforementioned objects, features and advantages of the present disclosure more obvious and understandable, the present disclosure will be further described below with reference to the accompanying drawings and embodiments.

It should be noted that specific details are set forth in the following description to fully understand the present disclosure. However, the present disclosure may be implemented in many other ways different from those described herein, and those skilled in the art may make similar generalizations without violating the connotation of the present disclosure. Therefore, the present disclosure is not limited by the specific embodiments disclosed below.

The terms used in the embodiments of the present disclosure are only for the purpose of describing specific embodiments and are not intended to limit the present disclosure. The singular forms of “a”, “said” and “the” used in the embodiments of the present disclosure and the appended claims are also intended to include plural forms, unless the context clearly indicates other meanings.

It should be noted that the example embodiments may be implemented in various forms, and should not be construed as being limited to the embodiments set forth herein. On the contrary, the provision of these embodiments makes the present disclosure more comprehensive and complete, and fully conveys the concept of the example embodiments to those skilled in the art. The same reference numerals in the figures indicate the same or similar structures, and thus their repeated description will be omitted. In addition, the similarities between the embodiments will not be repeated.

Disclosed herein include methods and systems for electrical and/or optical signal-based stress management by using a stress analysis pipeline that determines predicted stress levels of individuals by implementing a predictive model trained to predict the stress level of an individual based on electrical and/or optical signals obtained from the individual. In particular embodiments, the stress analysis pipeline determines the predicted stress level of an individual by implementing a predictive model trained to predict the stress level of an individual based on electroencephalogram (EEG), photoplethysmography (PPG), and/or remote PPG (rPPG), among other possible signals obtained from the individual, including but not limited to audio signals for measuring the individual's voice, tone of voice, and choice of words, and positioning signal for measuring the individual's movements, and so on. Based on these signals, the stress analysis pipeline may predict the stress level of an individual at any specific moment, for example, when the individual has the best stress levels and when the individual has the worst stress levels.

In some embodiments, the stress analysis pipeline disclosed herein is also configured to identify certain mental exercises for each stress level, which may include specific video, audio, image, or other proper content that may be presented to an individual for relieving the stress of the individual at the respective stress level.

In some embodiments, a series of mental exercises may be predefined or developed for each stress level, so that when an individual is identified to have stress at a specific level, one or more mental exercises may be selected from the predefined mental exercises and recommended to the individual. In some embodiments, to develop predefined mental exercises, for each detected stress level, a series of mental exercises may be first proposed by professional consultants (e.g., leading neuroscientists and mental wellness experts). An individual may be then subject to the proposed mental exercises (e.g., displaying video/audio/image to the individual for relieving stress). After practicing the mental exercises, the individual may be subject to stress level detection again by obtaining the electrical and/or optical signals from the individual and predicting the stress level by the trained predictive model. The stress level change may indicate whether the proposed mental exercises help relieve the stress experienced by the individual. In this way, a series of mental exercises may be developed for each stress level.

In some embodiments, the stress analysis pipeline disclosed herein is further configured to generate one or more recommendations after a stress level is identified for an individual by the trained predictive model. For example, one or more mental exercises may be selected from the series of mental exercises developed for the corresponding stress level. In some embodiments, the series of mental exercises are developed for the corresponding stress level based on the feedback from a plurality of different individuals. Accordingly, when selecting mental exercises for recommendation to a specific individual, the stress analysis pipeline may take into account the personal information such as the user preference of the user. This allows to generate a recommendation based on both the detected stress level and the personal information of the user. In some embodiments, the stress analysis pipeline may automatically take other factors not described above when generating a recommendation. In one example, the stress analysis pipeline may be coupled to an artificial intelligence (AI) engine when generating a recommendation based on the detected stress level and the personal information of the individual. The AI engine may generate or create recommendations by taking many additional factors into consideration (e.g., current trends in stress relief).

In some embodiments, the stress analysis pipeline disclosed herein is further coupled to a scheduler that allows to determine a proper schedule including proper tasks for an individual based on the stress levels detected for the individual, as will be described in detail later.

The stress analysis pipeline disclosed herein shows advantages over other existing stress management methods or systems. For example, in the disclosed stress analysis pipeline, electrical and/or optical signals are obtained and utilized to predict the stress level of an individual, instead of relying on personal observations as found in professional consultations. This signal-based stress prediction provides technical improvements when compared to other existing stress management methods as it helps with quickly detecting stress levels and managing these stressed conditions timely. Recent advancements in mobile technology allow certain electrical and/or optical signals to be obtained instantly and conveniently through wearable devices, which facilitates the instant detection of stress levels at any desired location, but not just limited to specific facilities as many other existing stress monitoring technology. In addition, in the stress analysis pipeline disclosed herein, a trained predictive model is utilized to detect the stress level for an individual, which is generally more accurate or subjective when compared to other existing stress monitoring technologies (e.g., professional consultations). Further, a recommendation pipeline may be integrated into or coupled to the stress analysis pipeline disclosed herein, which allows one or more mental exercises to be automatically and instantly generated and provided to the individual, so that a stressed condition may be timely addressed at a desired location without restriction, which may prevent catastrophic outcomes such as accidents, injuries, and even fatalities.

It is to be noted that the benefits and advantages described herein are not all-inclusive, and many additional features and advantages will be apparent to one of ordinary skill in the art in view of the figures and the following descriptions.

### Overall System

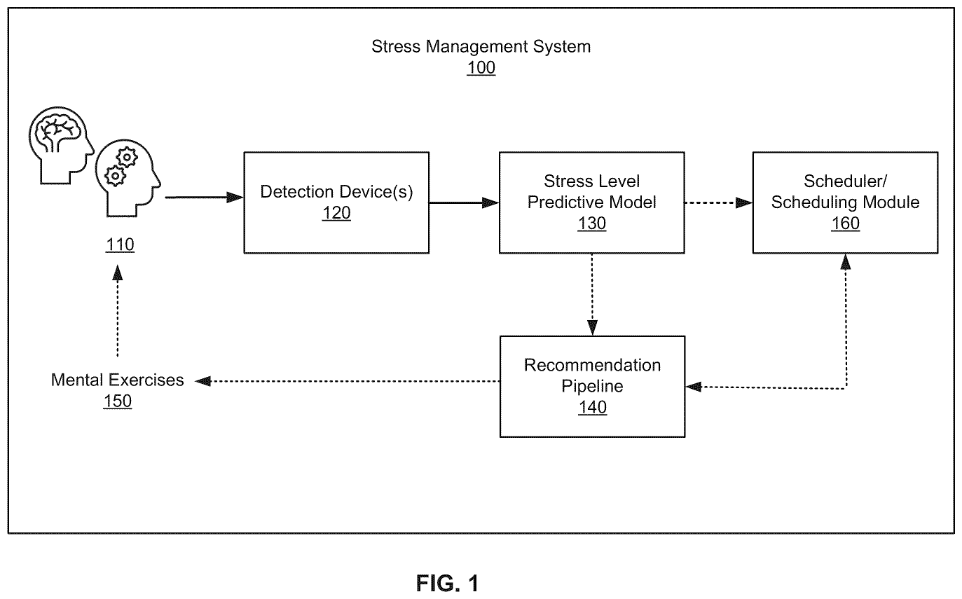

FIG. 1 shows an overall stress management system 100 for implementing a stress analysis pipeline, in accordance with an embodiment. Generally, the stress management system 100 includes one or more individuals 110 that are to be analyzed for stress. In some embodiments, an individual 110 may show a sign of stress, and thus it is desirable to determine the stress level experienced by the individual, so that proper action may be timely taken. In some embodiments, when an individual shows a sign of stress, certain physiological activities may reflect the stress experienced by the individual. For example, an individual under stress may show changes in electrical activity in the brain, heart rate variability (HRV), volumetric changes in blood in peripheral circulation, among other possible physiological changes experienced by the individual.

In some embodiments, various physiological changes experienced by an individual under stress may be measured by one or more detection devices 120 included in the stress management system 100. For example, electroencephalogram (EEG) is a test that measures electrical activity in the brain. The test uses small, metal discs called electrodes that attach to the scalp. Brain cells communicate via electrical impulses, and this activity shows up as wavy lines on an EEG recording. Brain cells are active all the time, even during sleep.

In some embodiments, EEG signals may be analyzed using Welch's fast Fourier transform (FFT) to extract power spectral density (PSD) features which represent the power of a signal distributed over a range of frequencies. Slow wave versus fast wave (SW/FW) of EEG may be utilized to discriminate stress from resting baseline. For example, the Alpha/Beta ratio and Theta/Beta ratio may be negatively correlated with stress, and thus the power ratios identified from the signals obtained from the EEG test may be utilized to discriminate the data characteristics of brainwaves for stress assessment. In some embodiment, the EEG signals with or without processing may be input into a predictive model for determining the stress level as will be described in detail later.

In some embodiments, a wearable EEG device may be configured and utilized to non-invasively record brain signals, where certain sensors (e.g., a number of dry electrodes, reference and common sensors) may be placed inside the wearable EEG device to measure the brain activities. In some embodiments, the signals obtained by the wearable EEG device may be converted into digital data before or after transferring to a wired or wirelessly (e.g., Bluetooth) connected device such as a smartphone, a tablet, or a computer for further analysis, for example, by a trained predictive model such as a stress level predictive model 130, as shown in FIG. 1. In some embodiments, EEG sensors may be integrated into another wearable device (e.g., a virtual reality device).

As another example, photoplethysmography (PPG) is an optical technique used to detect volumetric changes in blood in peripheral circulation. It is a low-cost and non-invasive method that takes measurements at the surface of the skin. PPG provides valuable information related to an individual's cardiovascular system. Recent advances in mobile or wearable technology have revived interest in this technique, which is widely used in clinical physiological measurement and monitoring, including the monitoring of stress. In one specific implementation, a one-dimensional (ID) PPG signal obtained by a PPG device can be transformed into 2D time-frequency images (e.g., scalograms) using continuous wavelet transform (CWT), which may be further fed into a predictive model for predicting the stress level, as will be described in detail later. In some embodiments, a wearable PPG device may be configured and placed around the wrist or in-car of an individual and utilized to non-invasively record optical signals reflecting volumetric changes in blood in the peripheral circulation. In some embodiments, signals obtained by a wearable PPG device may be converted into digital data before or after transferring to a wirelessly connected device such as a smartphone, a tablet, or a computer for further analysis, for example, by a trained stress level predictive model 130, as shown in FIG. 1. In some embodiments, one or more PPG sensors may be integrated into another wearable device, such as a virtual reality device.

In yet another example, rPPG is also an optical technique used to measure heart rate (HR) and HRV remotely without attaching sensors to individuals. HR is influenced by the autonomic nervous system which consists of two primary branches, the sympathetic and the parasympathetic nervous system. The former is active when people are in an exciting situation (threat, fear, stress, and exercise) and increases heart rate. The latter is active when people are in a relaxed situation (feelings of love, compassion, and calm) and decreases heart rate. HRV is the indicator of the balance of those conditions, being a marker of health and stress. When the sympathetic nervous system is predominant like when people feel stressed, HRV decreases. When the parasympathetic nervous system is predominant like when people are relaxed, HRV increases.

rPPG measures HR and HRV using a high-resolution recording camera, which can be a mobile device camera, a laptop camera, and the like. It is a contactless measurement that measures the variance of red, green, and blue light reflection changes from the skin, as the contrast between specular reflection and diffused reflection. Specular reflection is the pure light reflection from the skin, while diffused reflection is the reflection that remains from the absorption and scattering in skin tissue, which varies by blood volume changes. In a specific example, a short video capturing the face of an individual (e.g., a 10-second, 20-second, 30-second, or another short period of face scan) may be taken by a high solution camera, which is then used to determine HR/HRV and thus predict the stress level of the individual.

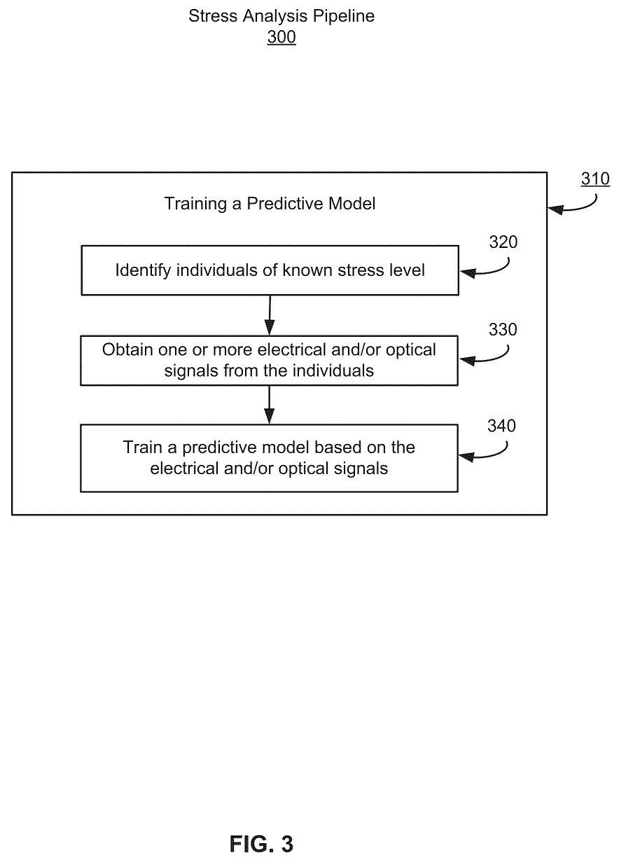

In the stress management system 100 disclosed herein, the stress level predictive model 130 may analyze electrical and/or optical signals obtained by the detection device(s) 102. For example, EEG, PPG, and/or rPPG data may be obtained from the wearable device(s) carried by an individual 110 or from a remote camera for a face scan of the individual, where the individual 110 may show a sign of stress (or may show no stress). In some embodiments, the stress level predictive model 130 may be trained first before being utilized for the prediction of the stress level of the individual, as will be described in detail in FIG. 3. For example, when properly trained, the stress level predictive model 130 may predict the stress level for the individual based on the detected electrical and/or optical signals, such as EEG, PPG, and/or rPPG signals.

In some embodiments, besides these EEG, PPG, and/or rPPG signals, the stress level predictive model 130 may also utilize other signals, such as an individual's voice, tone, choice of words, movements, and so on. In this way, the stress level predictive model 130 may build a forecast for an individual's stress level based on the user's circadian rhythm as measured by HR/HRV and many other measurements as described above.

In some embodiments, the stress management system 100 may additionally include a recommendation pipeline 140 configured to recommend certain mental exercises 150 for the detected stress level for an individual 110. For example, when a high level of stress is predicted by the stress level predictive model 130 based on the obtained electrical and/or optical signals from an individual, one or more mental exercises 150 may be recommended to the individual based on the predicted stress level. In some embodiments, a recommended mental exercise may be one of an image, audio, or video that may be presented to the individual in a two-dimensional (2D) environment, allowing the individual to relieve the stressed emotion.

In some embodiments, the mental exercises such as images, audios, or videos may be delivered to an individual 110 in a virtual (e.g., mixed or augmented) reality (VR) environment. For example, the individual may wear a VR device. Accordingly, in some embodiments, the delivered image, video, or audio may be generated in view of the mixed or augmented reality environment, allowing for proper delivery of the image, video, or audio for mental exercise for an individual wearing the VR device. In some embodiments, the EEG sensor(s) and/or PPG sensor(s) may become a part of the VR device wearable by the individual. In some embodiments, the stress level predictive model 130 and/or the recommendation pipeline 140 may be also a part of the VR device, or may be coupled to the VR device for stress level determination and for generating a proper recommendation based on the determined stress level. In some embodiments, the recommended mental exercises may be generated based on other user information. For example, if an individual 110 has Alzheimer's disease or memory loss problems, the recommended mental exercises 150 may be different when compared to an individual without such problems. Additional user information may include the user preference that may be taken into account in generating the proper recommendations.

In some embodiments, the recommendation pipeline 140 may be an AI engine or may be coupled to an AI engine, such as a generative AI that is configured to automatically generate one or more mental exercises 150. Generative AI or generative artificial intelligence refers to the use of AI to create new content, like text, images, music, audio, and videos. Generative AI is powered by foundation models (large AI models) that can multi-task and perform out-of-the-box tasks, including summarization, Q&A, classification, and more. Plus, with minimal training required, foundation models can be adapted for targeted use cases with very little example data. Generative AI works by using an ML model to learn the patterns and relationships in a dataset of human-created content. It then uses the learned patterns to generate new content. In the embodiments disclosed herein, the ML model may be a stress level predictive model 130 that is trained to learn the patterns from the EEG, PPG, and/or rPPG data and relationships between image/video/audio and the stress experienced by the individual 110, and thus generate or create the mental exercises 150 (e.g., videos, audios, or images) based on the patterns identified from the EEG data and/or PPG data.

In some embodiments, an individual 110 may be represented by an avatar in the VR environment, and the mental exercises recommended by the generative AI such as image, video, or audio may become interactive with the avatar representing the individual in the VR environment to allow the avatar to explore various images, audios and videos for mental exercise. In one example, objects and humans in an application scene in a specific location may be captured in video, audio, or image, which may be further mapped to a conceptual taxonomy that may be used to highlight the important idea of the application scene through the VR environment. In some embodiments, in such a VR environment, an end user may communicate with an internal avatar by speaking to the internal avatar (or an AI chatbot) for interactive actions in the VR environment. For example, an end user may speak to an internal avatar to play a video or audio. In some embodiments, there are many different avatars in the VR environment.

In some embodiments, to improve awareness and to encourage individuals to actively monitor their stress conditions, a reward program may be further included in the disclosed stress management system 100. The reward program may be configured to generate a reward when an individual participates in a mental exercise. The more mental exercises an individual participates in, the more rewards that the individual will get from the reward program. In some embodiments, rewards may be generated based on the stress level(s) experienced by an individual. A higher reward may be generated when an individual is in a better mood when the individual is assessed for the stress level.

Referring back to FIG. 1, in some embodiments, the stress management system 100 may optionally include a scheduler or a scheduling module 160 coupled to another third-party scheduling application) for task/schedule management for individuals. The scheduler or scheduling module 160 may collect task information (e.g., tasks input by a user) related to tasks to be performed by an individual. In some embodiments, the stress management system 100 may optionally include a large language model (LLM) that is configured to analyze the complexity of the tasks to be performed by the individual, and assign a value to the analyzed complexity. In some embodiments, the stress management system 100 may optionally integrate with various work related software (e.g., Jira, Confluence etc.) to extract the time it takes for various tasks and thus assess their complexities. In some embodiments, certain machine learning or AI models may be utilized to determine the complexity of tasks to be performed by an individual.

In some embodiments, the scheduler or scheduling module 160 may determine the proper schedule for an individual based on the detected stress levels for the individual and the complexity determined for various tasks. For example, the scheduler or scheduling module 160 may create a proper schedule for the individual where the best tasks are mapped to the times when the user is mentally and physically at best. This improves productivity for the individual. In some embodiments, the stress management system 100 may identify a daily/weekly stress fluctuation pattern by assessing the stress of the individual for a certain period of time (e.g., a week, a month, a few months, etc.). Based on the identified pattern, the stress management system 100 may determine an optimized schedule where the most complicated tasks are mapped to the times when the user is mentally and physically at best while less demanding tasks are scheduled to moments when an individual can not handle the stress well. In some embodiments, the scheduler or scheduling module 160 may use convex optimization and other optimization techniques to create a schedule.

In some embodiments, the scheduler or scheduling module 160 may adjust an already determined schedule of an individual based on the stress level detected for the individual. In some embodiments, the detection device(s) 120 (e.g., a wearable device such as a VR device) may consistently provide electrical and/or optical signals to the stress level predictive model 130, to allow the stress level predictive model 130 to assess the stress level in real-time. Based on the assessed stress level, the schedule of the individual may be timely adjusted or optimized if it is found the scheduled task does not match the determined stress level. For example, when performing a specific task following a schedule, if an individual is found to be not up to the mark in terms of mental or physical readiness for the task (e.g., based on the determined task complexity and stress level), the stress management system 100 may timely adjust the schedule of the individual.

In alternative embodiments, the stress management system 100 may identify appropriate mental exercises that can be scheduled in the allocated time, which will help improve the stress level of the individual (e.g., lower the stress level) so that the individual can then go ahead and do the remaining tasks. In an example application, an individual's stress is analyzed in real-time in a Facetime® kind of interaction (e.g., using PPG, voice, and/or movement signals), and an AI chatbot/avatar in a VR environment may start guiding the individual in the appropriate mental exercises to do for successfully completing various user tasks.

In some embodiments, the stress management system 100 disclosed herein not only assesses the stress for an individual, but also monitors a focus level, cognitive ability, alertness, attention, and the like, and takes these various factors into consideration when determining an optimal schedule for the individual. This may allow a true work-life balance to be achieved in a demanding world, and allow a person to manage their various day-to-day tasks in the most optimal way.

In some embodiments, when combined with educational or training material, the stress management system 100 disclosed herein may schedule various classes in a sequence that helps improve learning and retention for the subject matter. The retention ground truth for training the predictive model may be calculated using the above mentioned psychometric tests.

In some embodiments, an approximate prediction of the higher bound of an individual's productivity (e.g., grades in the case of students) may be further made by the disclosed stress management system 100. For example, the stress analysis pipeline may be configured to estimate an individual's maximum potential productivity (e.g., academic performance for students) by analyzing the interplay between mental/physical well-being, task complexity, and individual capabilities. In some embodiments, the assessment of the productivity gains due to mental and/or physical exercise and the complexity of a task may help educators design better individualized training curricula.

In some embodiments, the stress/focus values, along with the complexity of tasks, may help create more sophisticated LLMs that can output smarter individualized task breakdowns for their various tasks. These LLMs may also serve as a collective source of human memory, as well as a source of a specific sequence of tasks that enable various people to become a better version of themselves. For example, by analyzing an individual's current state (stress, focus, etc.) and the complexity of a given task, these LLMs may suggest optimal task breakdowns and sequences to help the individual achieve peak performance. This technology can be especially beneficial for complex projects that require careful planning and execution. In some embodiments, the LLMs may be further configured to simplify the interactions with individuals. In one example, the LLMs may be configured to summarize the outputs, such as physical and mental exercise recommendations and other recommendations (e.g., classes for improving a student's performance) based on user interactions. In some embodiments, the user journey including user interactions may be utilized to further train the LLM models for improved performance.

Altogether, by understanding the complex relationship between various factors that influence productivity, a new level of personalized learning and performance optimization may be unlocked by the disclosed stress management system 100. This approach has the potential to revolutionize education, work, and personal development, leading to a more productive and fulfilling life for everyone. Additional descriptions regarding the automated hardware and processes for stress management are described herein.

### Predictive Model System

Generally, a predictive model system (e.g., stress level predictive model 130 described in FIG. 1) analyzes physiological activity data of an individual 110, which is captured by the detection device(s) 120. In some embodiments, the predictive model system analyzes physiological activity data of a plurality of individuals for training a predictive model. In some embodiments, the predictive model system analyzes the physiological activity data of an individual for deploying a trained predictive model to predict the stress level of the individual. In some embodiments, the predictive model system and/or predictive models analyze the physiological activity data by at least analyzing values or features of the physiological activity data detected by the detection device(s) 120, e.g., by extracting values or the features from the physiological activity data or by deploying a neural network that extracts features from the physiological activity data in the form of a deep embedding vector as will be described in detail later.

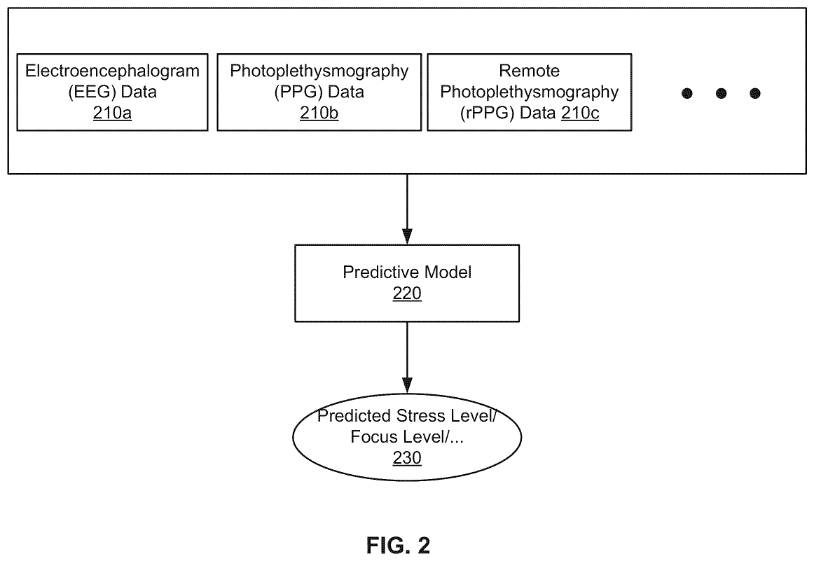

Reference is now made to FIG. 2, which is a block diagram that depicts the deployment of the stress level predictive model 130, in accordance with an embodiment. FIG. 2 shows EEG data 210a, PPG data 210a, and rPPG data 210c that may be obtained by the detection device(s) 120. For example, an individual 110 may wear a wearable device, which may include one or more sensors for detecting electrical signal (e.g., EEG data 210a) and/or optical signal (e.g., PPG data 210b) consistently or on demand. Additionally or alternatively, the individual 110 may perform a face scan by using a camera of a mobile device or a laptop, or by using a standalone webcam, which allows rPPG data 210c to be obtained based on the optical signal captured during the face scan. The electrical signal (e.g., EEG data 210a) and optical signal (e.g., PPG data 210b and rPPG data 210c), once detected by the detection device(s) 120, may be pre-processed or may be directly fed into a prediction model 220 (e.g., the stress level predictive model 130 shown in FIG. 1), which then predicts the stress level for the individual 110 based on the detected electrical and/or optical signals from the individual 110. The predicted stress level 230 is then obtained for the individual 110 in real-time. It should be noted that the detection device(s) 120 may detect other additional signals such as signals related to an individual's voice, tone, pronounced words, movements, and so on, the deployed stress level predictive model 130 may predict the stress level as well as focus level, cognitive ability, attention, alertness, and the like, if the model is properly trained before the deployment.

Reference is further made to FIG. 3, which is a block diagram that depicts a process 310 for training a stress level predictive model, in accordance with an embodiment. Briefly, the process 310 includes a step 320 of identifying individuals with known stress levels, a step 330 of obtaining one or more electrical and/or optical signals from each individual, and a step 340 of training a predictive model (e.g., the stress level predictive model 130) based on the electrical and/or optical signals obtained from the plurality of individuals of known stress levels.

As described above, the predictive model disclosed herein analyzes the electrical and/or optical signals from an individual and outputs a prediction of the stress level of the individual. In some embodiments, the predictive model may be any one of a regression model (e.g., linear regression, logistic regression, or polynomial regression), decision tree, random forest, support vector machine, Naïve Bayes model, k-means cluster, or neural network (e.g., feed-forward networks, multilayer perceptron networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In some embodiments, the predictive model comprises a dimensionality reduction component for visualizing data, the dimensionality reduction component comprising any of a principal component analysis (PCA) component or a T-distributed Stochastic Neighbor Embedding (TSNe). In some embodiments, the predictive model is a neural network. In some embodiments, the predictive model is a random forest. In some embodiments, the predictive model is a regression model, which is not limited in the present disclosure.

In some embodiments, the predictive model includes one or more parameters, such as hyperparameters and/or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, variables and threshold for splitting nodes in a random forest, support vectors in a support vector machine, and coefficients in a regression model. The model parameters of the predictive model are trained (e.g., adjusted) using the training data to improve the predictive power of the predictive model.

In some embodiments, the predictive model outputs a classification of the stress level of an individual. In one example, the stress may be classified into a number (e.g., 3, 5, 10, or another number) of levels. A higher level may indicate a higher stress, and a lower level may indicate a lower stress. In some embodiments, the predictive model outputs a score instead within a score range (e.g., 0-100 or another different range), where a higher score may indicate a higher stress and a lower score may indicate a lower stress.

In some embodiments, the predictive model disclosed herein may be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient descent, and dimensionality reduction techniques such as manifold learning, principal component analysis, factor analysis, autoencoder regularization, and independent component analysis, or combinations thereof. In particular embodiments, the predictive model is trained using a deep learning algorithm. In particular embodiments, the predictive model is trained using a random forest algorithm. In particular embodiments, the predictive model is trained using a linear regression algorithm. In some embodiments, the predictive model is trained using supervised learning algorithms, unsupervised learning algorithms, semi-supervised learning algorithms (e.g., partial supervision), weak supervision, transfer, multi-task learning, or any combination thereof.

In some embodiments, the predictive model disclosed herein is trained to improve its ability to predict the stress level of an individual using training data that include reference ground truth values that can be generated based on some psychometric tests for predicting the stress level, such as depression anxiety and stress scale (DASS) tests. For example, a reference ground truth value may be a known stress level of an individual. In a training iteration, the predictive model analyzes the detected electrical and/or optical signals from an individual and determines a predicted stress level of the individual. The predicted stress level of the individual may be compared against the reference ground truth value (e.g., known stress level of the individual) and the predictive model is tuned to improve the prediction accuracy. For example, the parameters of the predictive model are adjusted such that the predictive model's prediction of the stress level of the individual is improved. In particular embodiments, the predictive model is a neural network and therefore, the weights associated with nodes in one or more layers of the neural network are adjusted to improve the accuracy of the predictive model's predictions. In some embodiments, the parameters of the neural network are trained using backpropagation to minimize a loss function. Altogether, over numerous training iterations across different individuals, the predictive model is trained to improve its prediction of stress levels among different individuals.

In some embodiments, the predictive model disclosed herein is trained on features of images (e.g., PPG and EEG graphy/grams) acquired from individuals of known stress levels. Here, features may be waveform-related features such as peaks and frequencies. In some embodiments, features may be organized as a deep embedding vector. For example, a deep neural network may be employed that analyzes images to determine a deep embedding vector (e.g., a morphological profile). In some embodiments, at each training iteration, the predictive model is trained to predict the stress level using the deep embedding vector (e.g., a morphological profile).

In some embodiments, a trained predictive model includes a plurality of morphological profiles that define different stress levels. In some embodiments, a morphological profile for an individual of a specific stress level refers to a combination of values of features that define the specific stress level. For example, a morphological profile of a particular stress level may be a feature vector including values of features that are informative for defining the specific stress level.

In some embodiments, the predictive model disclosed herein may be trained to predict other values related to task performance. In one example, through a similar training process but based on a different set of training data, the predictive model may be trained to predict a focus level for an individual. For example, training data that include reference ground truth values for focus level determination can be generated based on some psychometric tests for predicting the focus, such as gradual-onset continuous performance task (GradCPT) tests. Other possible values that may be forecasted by the predictive model include but are not limited to the cognitive ability, attention, alertness, and the like, all of which may be properly predicted by the predictive model disclosed herein if the model is properly trained (e.g., trained by a properly prepared training data).

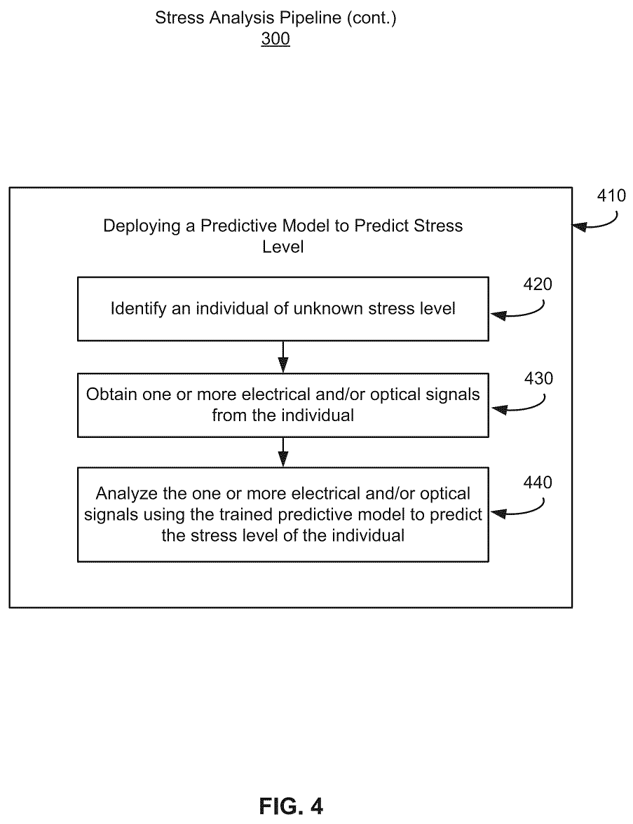

Reference is now made to FIG. 4, which is a block diagram that depicts a process 410 for deploying a predictive model to predict the stress level of an individual, in accordance with an embodiment. Briefly, the process 410 includes a step 420 of identifying an individual with an unknown stress level, a step 430 of obtaining one or more electrical and/or optical signals from the individual, and a step 440 of analyzing the one or more electrical and/or optical signals using the trained predictive model to predict the stress level of the individual. In some embodiments, the predictive model may be also configured to predict the focus level, cognitive ability, attention level, alertness level, and the like for the individual, as described earlier.

In some embodiments, a series of mental exercises (which may include videos, images and audios) may be predefined by the professionals. When the stress level of an individual is determined, one or more mental exercises corresponding to the detected stress level may be selected from the predefined mental exercises and provided to the individual for stress relief.

In some embodiments, new content (e.g., a new video, audio, image) having a potential to relieve stress may have been created by a creator (or by generative AI), but has not been evaluated and/or categorized by a professional or expert. At this moment, the predictive model disclosed herein may be utilized to evaluate the new content to determine whether it has the potential to relieve the stress, and the likely stress level that it may be applicable to. This can be achieved by the disclosed predictive model, even without input from a leading neuroscientist and mental wellness expert.

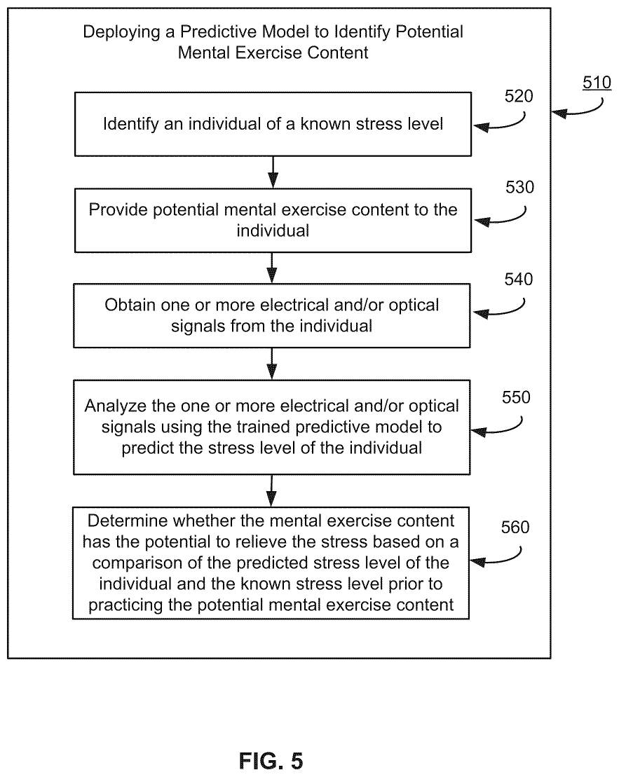

Reference is further made to FIG. 5, which is a block diagram that depicts a process 510 for deploying a predictive model to evaluate potential mental exercise content, in accordance with an embodiment. Briefly, the process 510 includes a step 520 of identifying an individual of a known stress level (which may be confirmed by the stress level predictive model), a step 530 of providing potential mental exercise content (e.g., a video, audio, or image) to the individual to allow the individual to practice the mental exercise, a step 540 of obtaining one or more electrical and/or optical signals from the individual after practicing the mental exercise (e.g., watching a video, audio, or image), a step 550 of analyzing the one or more electrical and/or optical signals using the trained predictive model to predict the stress level of the individual after practicing the mental exercise by the individual, and a step 560 of determining whether the mental exercise content has the potential to relieve the stress based on a comparison of the predicted stress level of the individual with the known stress level before the individual practices the mental exercise. If the predicted stress level is lower than the known stress level before practicing the potential mental exercise content, it is determined that the potential mental exercise content has the capacity to relieve the stress. At this moment, the potential mental exercise content may be added to the list of predefined mental exercises and may be recommended to the same or different individuals later. In some embodiment, a series of processes 510 may be further performed to determine the corresponding stress level to which the potential mental exercise content is applicable.

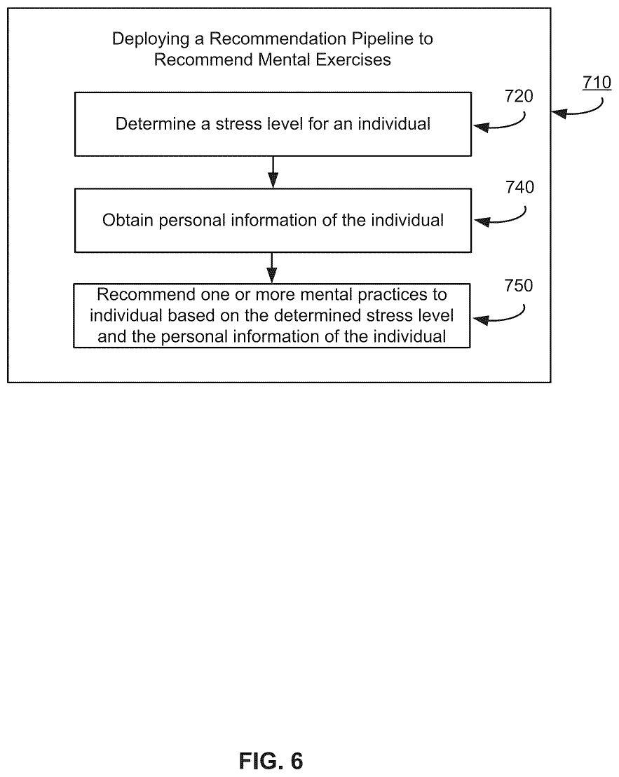

Reference is further made to FIG. 6, which is a block diagram that depicts a process 610 for recommending one or more mental exercises for an individual, in accordance with an embodiment. Briefly, the process 610 includes a step 620 of determining a stress level for an individual, a step 630 of obtaining personal information of the individual, and a step 640 of recommending one or more mental practices to the individual based on the determined stress level and the personal information of the individual.

In some embodiments, a recommendation pipeline 140 is included in the disclosed stress management system 100 and is configured to generate one or more mental exercises for recommendation to an individual after the stress level of the individual is determined. In some embodiments, the stress level of the individual is determined by the stress level predictive model 130 and the recommendation pipeline 140 is coupled to the stress level predictive model 130 and determines one or more mental exercises for recommendation right after the stress level for the individual is determined by the stress level predictive model 130. In some embodiments, there is a series of mental exercises predefined for each stress level. When the stress level of an individual is determined, one or more mental exercises may be selected from the series of mental exercises corresponding to the determined stress level.

In some embodiments, the recommendation pipeline 140 may take into account additional information when making the recommendations. For example, the personal information of the individual is utilized to determine the proper mental exercises for recommendation. In a specific example, if an individual prefers video over audio or image, one or more videos may be selected from the series of mental exercises corresponding to the determined stress level. In another specific example, if an individual is a kid or youth, one or more mental exercises proper for the kids or youths (e.g., fun cartoons) may be recommended to the individual.

In some embodiments, the recommendation pipeline 140 included in the stress management system 100 may be coupled to an AI engine such as a generative AI, which allows intelligent recommendations to be generated and/or provided to an individual. For example, the determined stress level of an individual and personal information of the individual may be submitted to a generative AI with a request to recommend mental exercises for stress relief. In response, the generative AI may provide one or more mental exercises to the individual based on the stress level and personal information, among other possible factors that may be considered by the generative AI. In one example, the generative AI may track the current trends in relieving stress, and recommend one or more mental exercises based on the current trends. In another example, the generative AI may track user activities and determine the user preference based on the tracked user activities, and then recommend one or more mental exercises based on the determined user preference or user habits. In yet another example, the generative AI may generate or create mental practices based on the determined stress level and personal information and deliver the generated mental exercises to an individual for recommendation.

In some embodiments, the recommendation pipeline 140 in the disclosed stress management system 100 does not just recommend mental exercises to an individual under stress, but may make other additional recommendations, such as rest, physical exercises, or appropriate diet and nutrition so that the individual can perform optimally.

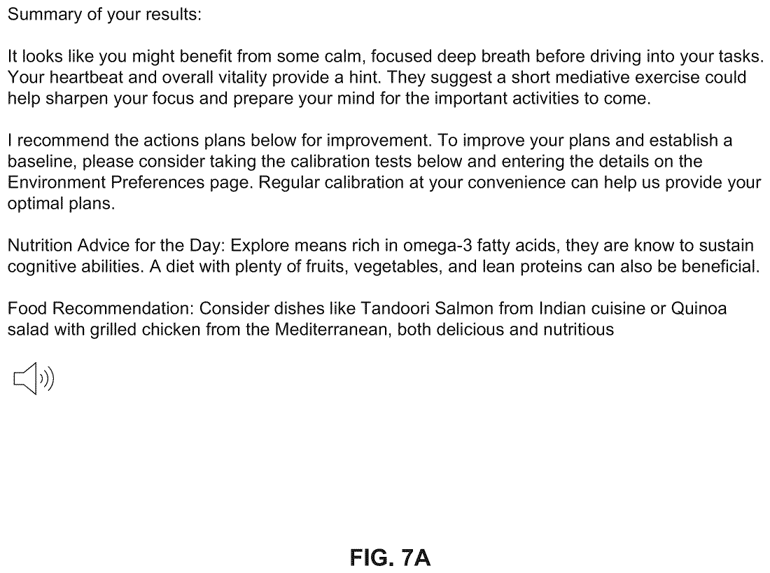

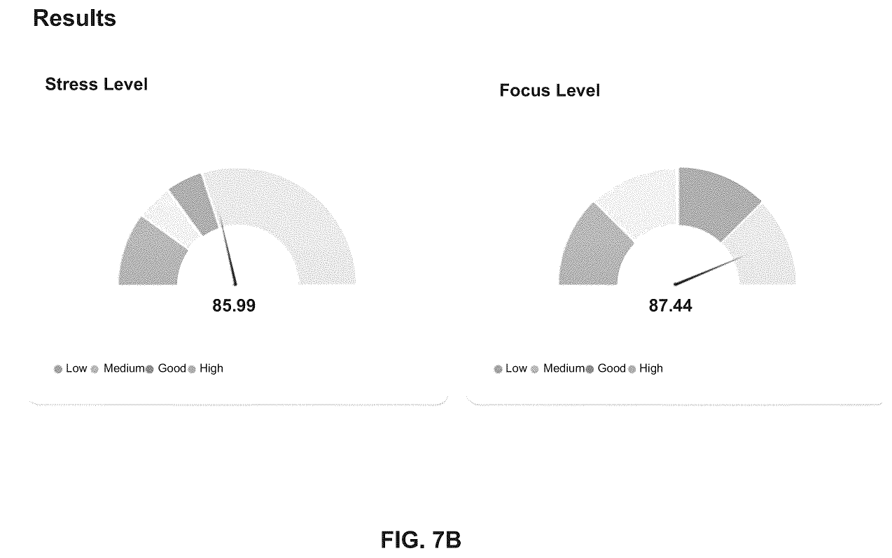

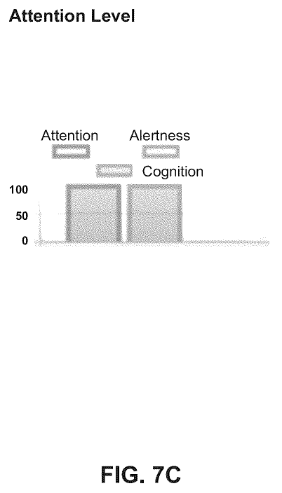

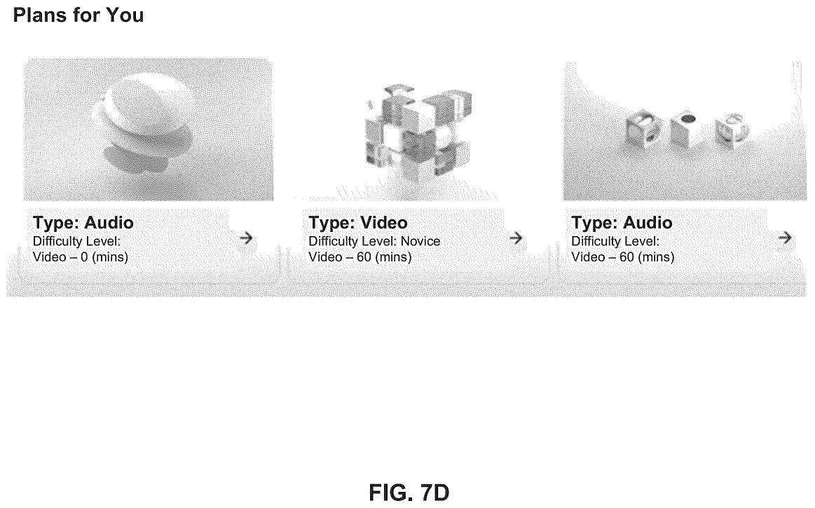

Reference is now made to FIGS. 7A-7D, which illustrate various outputs generated by the disclosed stress management system 100, in accordance with an embodiment. Specifically, FIG. 7A illustrates a summary of the results, which may be automatically generated by one or more LLMs described above, indicating the detected stress level, focus level, cognitive ability, or the like, and certain recommendations determined based on the detected stress level, focus level, cognitive ability, or the like. As can be seen from FIG. 7A, recommendations may include certain mental exercises, diet, and nutrition recommendations. As also illustrated in FIG. 7A, a speaker may allow an individual to choose to listen to these results or recommendations. FIGS. 7B-7C further illustrates some specific results, such as the detected stress level, focus level, and attention level, which further includes the detected attention, alertness, and cognition levels. FIG. 7D also illustrates the recommended mental exercises, such as specific videos and/or audios.

It should be noted that only some example outputs are provided in FIGS. 7A-7D. In some embodiments, the disclosed stress management system 100 may generate additional outputs, such as cumulative stress level, cumulative focus level, cumulative depression, anxiety, and stress, etc. In addition, an additional user interface may be also generated for scheduling one or more tasks for an individual, including receiving user inputs for specific tasks, and automated mapping of the tasks to the calendar based on the assessed complexity and determined stress levels at various time points (which can be predicted based on the historical information of the individual).

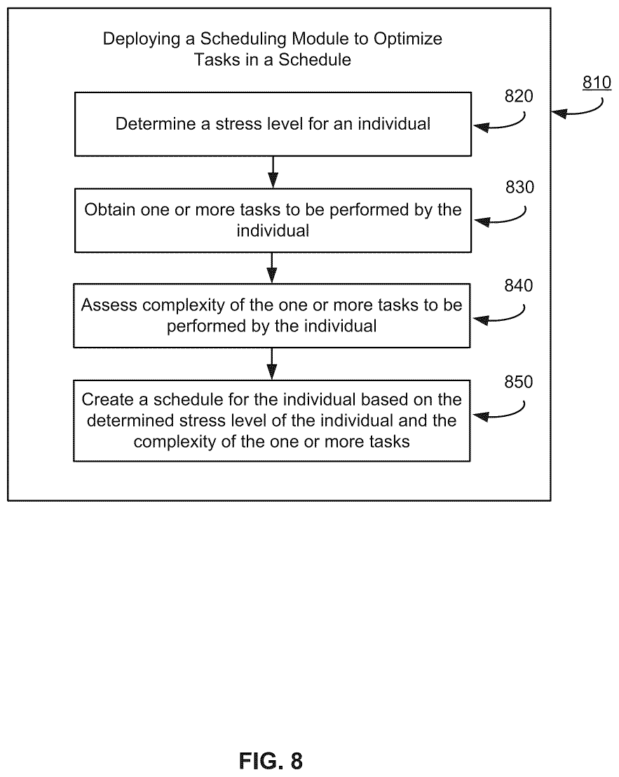

Reference is further made to FIG. 8, which is a block diagram that depicts a process 810 for creating a proper schedule for an individual, in accordance with an embodiment. Briefly, the process 810 includes a step 820 of determining a stress level for an individual, a step 830 of obtaining tasks to be performed by the individual, a step 840 of assessing the complexity of the tasks to be performed by the individual, and a step 850 of creating a proper schedule for performing the tasks by the individual based on the determined stress level and the complexity of the tasks.

In some embodiments, the stress level of an individual at different moments may be predicted in advance based on the historical stress information of the individual monitored by the stress management system 100. For example, the historical stress information of the individual may indicate that the individual is mentally and physically at best during early morning hours, and thus complicated tasks are scheduled for early morning hours. This improves the productivity of the individual.

In some embodiments, when creating the proper schedule for an individual, the focus level, cognitive ability, attention, alertness, and other factors that affect the task performance are also considered, so that the created schedule balances work and life, which allows a person to manage their various day-to-day tasks in the most optimal way.

### System and/or Computer Embodiments

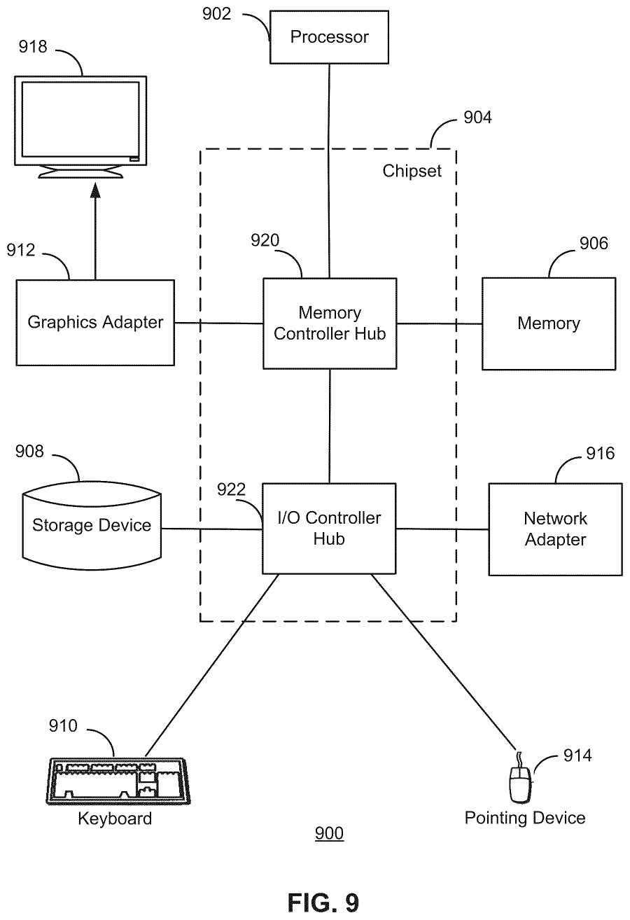

FIG. 9 depicts an example computing device 900 for implementing systems and methods described in reference to FIGS. 1-8. Examples of a computing device may include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like. In some embodiments, the computing device 900 may operate as the predictive model system 130 shown in FIG. 1 (or a portion of the predictive model system 130). Thus, the computing device 900 may train and/or deploy predictive models for predicting disease states of individuals.

In some embodiments, the computing device 900 includes at least one processor 902 coupled to a chipset 904. The chipset 904 includes a memory controller hub 920 and an input/output (I/O) controller hub 922. A memory 906 and a graphics adapter 912 are coupled to the memory controller hub 920, and a display 918 is coupled to the graphics adapter 912. A storage device 908, an input interface 914, and network adapter 916 are coupled to the I/O controller hub 922. Other embodiments of the computing device 900 have different architectures.

The storage device 908 is a non-transitory computer-readable storage medium such as a hard drive, compact disk read-only memory (CD-ROM), DVD, or a solid-state memory device. The memory 906 holds instructions and data used by the processor 902. The input interface 914 is a touch-screen interface, a mouse, track ball, or other type of input interface, a keyboard, or some combination thereof, and is used to input data into the computing device 900. In some embodiments, the computing device 900 may be configured to receive input (e.g., commands) from the input interface 914 via gestures from the user. The graphics adapter 912 displays images and other information on the display 918. The network adapter 916 couples the computing device 900 to one or more computer networks.

The computing device 900 is adapted to execute computer program modules for providing functionality described herein. As used herein, the term “module” refers to computer program logic used to provide the specified functionality. Thus, a module may be implemented in hardware, firmware, and/or software. In one embodiment, program modules are stored on the storage device 908, loaded into the memory 906, and executed by the processor 902.

The types of computing devices 900 may vary from the embodiments described herein. For example, the computing device 900 may lack some of the components described above, such as graphics adapters 912, input interface 914, and displays 918. In some embodiments, a computing device 900 may include a processor 902 for executing instructions stored on a memory 906.

The methods disclosed herein may be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of this disclosure. Such data may be used for a variety of purposes, such as patient monitoring, treatment considerations, and the like. Embodiments of the methods described above may be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in a known fashion. The computer may be, for example, a personal computer, microcomputer, or workstation of conventional design.

Each program may be implemented in a high-level procedural or object-oriented programming language to communicate with a computer system. However, the programs may be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

The signature patterns and databases thereof may be provided in a variety of media to facilitate their use. “Media” refers to a manufacture that contains the signature pattern information of the present disclosure. The databases of the present disclosure may be recorded on computer readable media, e.g., any medium that may be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art may readily appreciate how any of the presently known computer readable mediums may be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats may be used for storage, e.g., word processing text file, database format, etc.

What is claimed is:

1. A method comprising:
identifying an individual with an unknown stress level;
obtaining one or more electrical or optical signals associated with stress from the individual;
predicting a stress level of the individual based on the one or more electrical or optical signals obtained from the individual, wherein the stress level of the individual is predicted by a predictive model; and
generating a recommendation for the individual based on the predicted stress level, the recommendation including one or more mental exercises for relieving stress for the individual.

2. The method of claim 1, wherein the one or more electrical or optical signals include one or more of an electroencephalogram (EEG) signal, a photoplethysmography (PPG) signal, or a remote PPG (rPPG) signal.

3. The method of claim 1, wherein the one or more electrical or optical signals include one or more signals obtained from a wearable device, the wearable device including one or more sensors configured to measure the one or more signals.

4. The method of claim 4, wherein the wearable device is a virtual reality (VR) device.

5. The method of claim 1, wherein the one or more electrical or optical signals includes a signal obtained from a camera, the camera performing a face scan of the individual to obtain the signal.

6. The method of claim 1, wherein the one or more mental exercises include one or more of an image, an audio, or a video.

7. The method of claim 1, wherein the one or more mental exercises are played in a two-dimensional environment when the individual practices the one or more mental exercises.

8. The method of claim 1, wherein the one or more mental exercises are played in a virtual reality environment when the individual practices the one or more mental exercises.

9. The method of claim 1, wherein the one or more mental exercises include a mental exercise generated by a generative artificial intelligence (AI).

10. The method of claim 1, further comprising:
obtaining one or more tasks to be performed by the individual;
assessing complexity of the one or more tasks to be performed by the individual; and
creating a schedule for performing the one or more tasks by the individual based on the determined complexity of the one or more tasks and the determined stress level of the individual.

11. The method of claim 10, further comprising:
creating one or more large language models (LLMs), the LLMs configured to automatically output breakdowns for the one or more tasks based on the determined complexity of the one or more tasks and the determined stress level of the individual.

12. The method of claim 11, wherein the one or more LLMs are configured to automatically generate a summary for the output breakdowns.

13. The method of claim 10, wherein the predictive model is further trained to predict one or more of a focus level, a cognitive ability, an attention level, or an alertness level for the individual.

14. The method of claim 13, wherein the schedule of the individual is created based on the predicted one or more of the stress level, focus level, cognitive ability, attention level, or alertness level for the individual.

15. The method of claim 1, wherein the one or more recommendations include one or more physical exercises, diet, or nutrition for the individual.

16. The method of claim 1, wherein the one or more recommendations include one or more classes for the individual, the one or more classes being arranged in sequence when more than one class is included in the recommendations.

17. A system, comprising:
a processor; and
a memory, coupled to the processor, configured to store executable instructions that, when executed by the processor, cause the processor to perform operations comprising:
identifying an individual with an unknown stress level;
obtaining one or more electrical or optical signals associated with stress from the individual;
predicting a stress level of the individual based on the one or more electrical or optical signals obtained from the individual, wherein the stress level of the individual is predicted by a predictive model; and
generating a recommendation for the individual based on the predicted stress level, the recommendation including one or more mental exercises for relieving stress for the individual.

18. The system of claim 17, wherein the one or more electrical or optical signals include one or more of an electroencephalogram (EEG) signal, a photoplethysmography (PPG) signal, or a remote PPG (rPPG) signal.

19. The system of claim 17, wherein the one or more electrical or optical signals are obtained from one or more of a wearable device or a camera.

20. The system of claim 17, wherein the operations further comprise:
obtaining one or more tasks to be performed by the individual;
assessing complexity of the one or more tasks to be performed by the individual; and
creating a schedule for performing the one or more tasks by the individual based on the determined complexity of the one or more tasks and the determined stress level of the individual.
